(19) United States
(12) Patent Application Publication
(10) Pub. No.: US 2023/0349902 A1
(43) Pub. Date: Nov. 2, 2023

(54) NOVEL T CELLS AND THE USES THEREOF

(71) Applicants: COMMISSARIAT A L'ENERGIE ATOMIQUE ET AUX ENERGIES ALTERNATIVES, PARIS (FR); HOPITAL FOCH, SURESNES (FR); ASSISTANCE PUBLIQUE - HÔPITAUX DE PARIS, PARIS (FR)

(72) Inventors: NATHALIE ROUAS-FREISS, PARIS (FR); JOEL LEMAOULT, MELUN (FR); EDGARDO CAROSELLA, PARIS (FR); OLIVIER BRUGIERE, PARIS (FR); DOMITILLE MOUREN, PARIS (FR)

(21) Appl. No.: 18/006,883

(22) PCT Filed: Jul. 26, 2021
(86) PCT No.: PCT/EP2021/070789

§ 371 Date: Jan. 26, 2023

(30) Foreign Application Priority Data
Jul. 27, 2020 (EP) 20305861.5

(51) Int. Cl. G01N 33/569; G01N 33/50; C12N 5/0783

(52) U.S. Cl. CPC G01N 33/56972; G01N 33/505; C12N 5/0637; G01N 2800/245

(57) ABSTRACT

The invention relates to a new population of T cells, expressing the following biomarkers: CD3, CD4, CD57 and ILT2, as well to the uses thereof, such as for diagnosis, prognosis or treatment of various immune pathologies or responses, including graft rejection, autoimmune diseases, infectious diseases or cancers.

### CROSS-REFERENCE TO RELATED APPLICATION

This application is the U.S. national stage application of International Patent Application No. PCT/EP2021/070789, filed Jul. 26, 2021.

### FIELD OF THE INVENTION

The invention relates to a new population of T cells, and to the uses thereof, such as for diagnosis, prognosis or treatment of various immune pathologies or responses, including graft rejection, autoimmune diseases, infectious diseases or cancers.

### BACKGROUND OF THE INVENTION

Lung transplantation (LTx) is now a validated treatment for end-stage respiratory failure. The main indications are cystic fibrosis, chronic obstructive bronchopneumopathy diseases (COPD), pulmonary fibrosis and pulmonary arterial hypertension (Weill et al, 2015). Despite advances in surgery and immunosuppressive therapy, post-transplantation mortality remains high, with a median survival of 5.7 years (Meyer et al. 2014).

Chronic Lung Allograft Dysfunction (CLAD), considered to be the equivalent of chronic rejection, is the primary cause limiting the long-term survival of lung transplant patients. The main phenotype of CLAD is bronchiolitis obliterans (BO), which accounts for 80% of CLAD cases, and reaches approximately 50% of patients at 5 years post-transplantation (Meyer et al. 2014). It is defined by a persistent decline in the maximum volume exhaled per 1 second (FEV1), of more than 20% compared to baseline FEV1, and after removal of other obvious causes of decline in FEV1 (such as bronchial stenosis, acute pneumonitis, acute rejection, etc.) (Verleden et al, 2019). The second phenotype is a restrictive form of CLAD called Restrictive Allograft Syndrome (RAS), identified in the last decade, affecting approximately 20% of patients (Glanville et al, 2019). It has recently been redefined by the association of a more than 20% decline in FEV1, and a more than 10% in Total Pulmonary Capacity (CPT) baseline post-Tx values, associated with the presence of persistent parenchymal scanner opacities (Glanville et al, 2019).

The pathogenesis of bronchiolitis obliterans (BO) includes a series of attacks of the bronchial epithelium, alloimmune or not, such as viral infections or acute rejection episodes, causing activation of the graft recipient's T lymphocytes by cells presenting graft antigens. This alloimmune response, specific to the graft antigens, ultimately leads to an aberrant bronchiolar repair that causes fibro-proliferation with a gradual reduction in the size of the airways (Verleden et al, 2019; Neuringer et al, 2005). Among the validated treatments of BO, macrolides are currently the only treatment that has led to incidence decrease (Verleden et al, 2019) and is used in preventive or curative treatment, depending on the centers. Other treatments have an uncertain level of evidence of efficacy, and include: increased intensity of immunosuppressive treatment, bolus IV of corticosteroids, anti-lymphocyte serum, extracorporeal photopheresis, and more recently treatment for desensitization during antibody-mediated rejection (Plasmapheresis, Immunoglobulin IV, Rituximab) (Verleden et al, 2019). Nevertheless, these treatments are often ineffective in slowing or stabilizing respiratory decline because they are administered at too late stage of the chronic rejection process. Thus, the only current treatment remains re-transplantation and concerns about 5% of lung transplants, thus reserved for hyper-selected candidates (Verleden et al, 2019).

Spirometric functional impairment, on which the diagnosis of BO is based, is a late event during the development of lesions, and therefore is of limited clinical interest because the fibro-proliferative bronchiolar lesions already present at this stage are irreversible (Neuringer et al, 2005). With regard to chronic restrictive rejection (RAS), a predominant role of humoral immunity is assumed, and de-immunization and anti-fibrotic treatments are currently under evaluation (Glanville et al, 2019).

There is currently no validated early marker predicting the occurrence of CLAD in pulmonary transplant patients. The diagnosis of CLAD is a functional one, based on the occurrence of a persistent decline in the maximum volume exhaled per 1 second (FEV1) to less than 80% of the basal value post-transplantation (Verleden et al, 2019). This diagnosis comes late in the process of development of intrapulmonary histological lesions of CLAD because at that time, the fibro-proliferative lesions of the terminal bronchioles are already irreversible. In addition, routine post-transplant transbronchial biopsy surveillance programs were not useful for the early diagnosis of CLAD because of frequent false-negative histology results, and the invasive nature of this method.

It is therefore crucial today to identify early and non-invasive biomarkers, which are predictive of the occurrence of CLAD. To date, no marker of this type is currently validated and used clinically. The early identification of these patients at risk of CLAD at a later stage would make it possible to adapt their immunosuppressive treatments upstream to stop this subclinical process.

Among the candidate markers predictive of the subsequent pulmonary function, several types of lymphocyte populations have already been studied in lung transplantation. First, the CD4+CD25hiCD127lo regulatory T cells (TREG), producing immunoregulatory cytokines such as TGFbeta and IL-10, were studied in organ transplantation, with conflicting results in lung transplantation (Salman et al., 2017; Meloni et al., 2006; Durand et al., 2018). Second, we previously reported increased expression of human leukocyte antigen G (HLA-G) in the bronchial epithelium of some lung transplantation recipients, which was associated with a stable condition at the date of biopsies (Brugiere et al., 2009), and we assessed the role of HLA-G expression as a predictor of graft acceptance (Brugiere et al., 2015).

In renal transplantation, cytotoxic CD4+CD57+ T cells have been reported to be associated with the risk of rejection resistant to Belatacept (recombinant CTLA4 immunoglobulin, blocking CD28-mediated costimulatory lymphocytes (Espinosa et al, 2016).

However, there is still a need to identify non-invasive optimized biomarkers, predictive of the occurrence of a CLAD, in order to identify early at-risk patients, and adapt the immunosuppressive treatments as early as possible, and avoid the development of low-level rejection lesions.

More generally, it is also necessary to develop optimized methods for diagnosis, prognosis and treatment of subjects who undergo organ transplantation or subjects who have other immune pathologies such as autoimmune diseases, infectious diseases or cancers.

### SUMMARY OF THE INVENTION

The present invention stems from the identification of a novel human T cell sub-population. More specifically, the invention stems from the identification, isolation and characterization of human T cells with the following phenotype CD3+CD4+CD57+ILT2+. The inventors have identified and isolated such cells from biological samples of human patients with pathological conditions and/or following organ transplantation. The inventors have shown such cells exhibit cytotoxic effect and also express the tolerogenic molecule ILT2, thus displaying unprecedented phenotype. The inventors have shown such cells can be correlated to inappropriate immune responses in human subjects. Such cells represent, inter alia, a marker of pathological conditions, as well as a candidate medicament or target for drug development. Modulators of such cells also represent new therapeutic drugs.

A first object of the invention relates to a method of monitoring a subject, comprising determining the presence or level of CD3+CD4+CD57+ILT2+ T cells in a sample from the subject.

The method may be used for monitoring a subject with an immune-related disorder, such as a subject who is transplanted or is a candidate for transplantation, a subject with a cancer, an autoimmune disease or an infectious disease, for instance. The subject is preferably a human.

A further object of the invention is a method for evaluating the risk of rejection of a transplant in a subject who is transplanted or is a candidate for transplantation, comprising determining, in a biological sample of the subject, the level of CD3+CD4+CD57+ILT2+ T cells, said level being indicative of the risk of transplant rejection.

A further object of the invention is a method for producing or selecting modulators of T cells, comprising exposing CD3+CD4+CD57+ILT2+ T cells to a test molecule or treatment, and determining whether the test molecule modulates an activity of said cells, preferably a cytotoxic activity of said cells.

A further object of the invention is a composition comprising CD3+CD4+CD57+ILT2+ T cells and an excipient or diluent.

A further object of the invention resides in a modulator of CD3+CD4+CD57+ILT2+ T cells for use as a medicament, particularly for the treatment of an immune disorder or inappropriate immune response.

A further object of the invention is a use of an inhibitor or depletion of CD3+CD4+CD57+ILT2+ T cells for the preparation of a medicament to reduce the risk of transplant rejection.

A further object of the invention is a kit comprising an ILT2 binding reagent and a CD57 binding reagent, preferably packaged in different containers. The kit may further comprise reagents for an immunological detection test and, possibly, an instruction manual. Preferably, the binding reagents are antibodies.

### BRIEF DESCRIPTION OF THE DRAWINGS

FIGS. 1A-1B: Freedom from CLAD and graft survival according to % of CD3+CD4+ILT2+CD57+ T cells at 1-month post-LTx, and to the 1-12 months CD3+CD4+ILT2+CD57+ T cells ratio. (FIG. 1A) the incidence of CLAD according to a cut-off of mean % of CD3+CD4+ILT2+CD57+ T cells at 1-month post-LTx (< or ≥ first IQR (25%), among the 107 patients with available 1-mo Time-point (log-rank test, p=0.04); (FIG. 1B) the incidence of CLAD according to increase of 1-12 months CD3+CD4+ILT2+CD57+ T cells RATIO (log-rank, p=0.014).

FIGS. 2A-2B: Demonstration of (FIG. 2A) cytotoxic function of CD3+CD4+CD57+ILT2+ T cells from healthy donor, or two patients LTx 7044V2 and LTx 6007V2: The monocytic cell line THP1 (ATCC) was used as target cells facing either CD3+CD4+CD57−ILT2− effector cells (shown as ‘ILT2-’) or CD3+CD4+CD57+ILT2+ effector cells (shown as ‘ILT2+’) from healthy donor or from two patients LTx 7044V2 and LTx 6007V2; and (FIG. 2B) inhibition of this cytotoxic function by HLA-G: THP1 target cells were transduced, or not, to express membrane-bound HLA-G1 (THP1 and THP1-HLA-G1 cells), as previously described (Dumont et al. Cancer Immunology Research, 2019). THP1 and THP1-HLA-G1 cells were used as target cells facing either CD3+CD4+CD57−ILT2− or CD3+CD4+CD57+ILT2+ effector cells.

FIG. 3: Example of cytograms identifying cell population CD3+CD4+CD57+ILT2+. An expression measured by flow cytometry of the different surface markers on CD4+ cells. Analyzes were performed from patient LTx 10005V3.

### DETAILED DESCRIPTION OF THE INVENTION

The invention relates to the identification of a new sub-population of T cells and to the uses thereof, e.g., for diagnosis, prognosis or treatment of various immune pathologies such as graft rejection, autoimmune diseases, infectious diseases or cancers.

In the present application, the inventors aimed, in particular, to investigate the potential role of the inhibitory checkpoint HLA-G/ILT-2 expressed by different peripheral blood cell populations in predicting subsequent chronic rejection-onset in LTx-recipients. For this purpose, peripheral blood T cells from a multicenter cohort of LTx recipients were analysed by flow cytometry for their cell-surface markers at predefined post-transplant time-points. Analysis was focused on the ILT-2 receptor of HLA-G, especially when associated to other markers known or suspected to be linked to graft stability and/or CLAD development, including CD57, PD1, CD25, CD127lo T-cell markers. ILT-2 is the only HLA-G receptor expressed by peripheral blood lymphocyte effectors. In healthy donors, approximately 20% of peripheral blood CD8+ T cells express ILT-2, and this proportion may increase with age, whereas % of CD4+ T cells expressing ILT-2 is currently unknown.

Remarkably and surprisingly, the inventors have identified an early increasing level of CD3+CD4⁺CD57⁺ILT2⁺ T cells within the first year in LTx recipients, which is associated with subsequent development of CLAD. This novel cell population represents a non-invasive predictor test for stratifying the risk of CLAD onset in LTx recipients. In addition, further functional experiments showed that these CD3+CD4+CD57+ILT2+ T cells exhibit cytotoxic properties, which demonstrate the relevance of these cells in other pathological conditions such as immune dysregulations, infectious diseases or cancers.

### Characterization of a New T Cell Population

In the context of their studies on transplant rejection, the inventors thus have identified a new population of CD4⁺ T cells, expressing the following membrane biomarkers: CD3, CD4, CD57, and ILT2. The combination of such markers is unprecedented and characterizes T cells with a unique phenotype, i.e., CD3+CD4+CD57+ILT2+.

CD3 (cluster of differentiation 3) is a T cell a co-receptor helping to activate both the cytotoxic T cells and T helper cells. CD4 (cluster of differentiation 4) is a glycoprotein present on the surface of immune cells such as T helper cells, monocytes, macrophages and dendritic cells.

CD57 (cluster of differentiation 57) is a glycoprotein expressed on NK cells, T cell subsets and some cells of neuroectodermal origin (Naeim et al., 2013). The proportion and absolute number of CD57-positive cells in peripheral blood increases with age. In adults, CD57 is expressed by 10-25% of the peripheral blood mononuclear cells. Most of the CD57+ T cells are of cytotoxic/suppressor type. Only a small fraction of CD57+ T cells expresses CD57, and these appear to be associated with chronic inflammatory conditions. The CD4+CD57+ cells are increased in lymphocyte predominance Hodgkin lymphoma and chronic inflammatory conditions. CD57 is a helpful marker for the detection of large granular lymphocytic (LGL) leukemia and is also positive in a wide variety of tumors of neuroectodermal or mesenchymal origin (Naeim et al., 2013).

ILT2 (immunoglobulin-like transcript 2) is an inhibitory receptor which is broadly expressed by B cells, T cells, NK cells, dendritic cells and other immune cells (Cosman et al., 1997). ILT2 interacts with MHC class I molecules such as HLA-E, HLA-F and HLA-G (Liang et al., 2006; Navarro et al., 1999). Interestingly, ILT2 binds to the immune-tolerogenic HLA-G molecule with a three- to four-fold higher affinity than to classical MHC class I molecules (Shiroishi et al., 2003). ILT2 contains in its cytoplasmic domain four immunoreceptor tyrosine-based inhibition motifs (ITIMs) that are involved in negative signaling through the recruitment of Src homology 2 (SH2) domain-containing proteins, such as SHP-1 (Bellon et al., 2002). The interaction of ILT2 with its ligand impairs the function and effector activity of T cells and B cells (Saverino et al., 2000; Naji et al., 2014). Thus, engagement of ILT2 on T cells has been shown to inhibit T cell antigen receptor (TCR) signaling and downstream events such as actin reorganization (Dietrich et al., 2001).

The new population of CD3+CD4+CD57+ILT2+ T cells have very unique combination of four molecules as described above.

In a first embodiment, the invention relates to these CD3+CD4+CD57+ILT2+ T cells. In this regard, the CD3+CD4+CD57+ILT2+ T cells according to the invention may be stored in any culture medium or any buffer solution, known in the art for storing T cells.

In particular embodiments, CD3+CD4+CD57+ILT2+ T cells of the invention may be in an isolated form, a purified form or mixed with one or more other cell populations. In a further embodiment, CD3+CD4+CD57+ILT2+ T cells of the invention may be genetically modified by any recombination technique known per se in the art.

Preferred cells of the invention are human. They may, alternatively, be derived from other mammalian species.

The inventors have, for the first time, identified such CD3+CD4+CD57+ILT2+ T cell sub-population with a unique phenotype, from samples of transplanted human subjects. The inventors have shown such population increases together with the risk of transplant rejection. The inventors have demonstrated such cells exhibit cytotoxic activity, and thus contribute to graft rejection. The inventors have further demonstrated such cells are present in cancer patients, where they may alter treatment efficacy and/or improve cancer escape from an immune response. These cells are generally absent (or present in small amounts) in healthy subjects.

Therefore, these new T cells thus constitute (i) a new biomarker as well as (ii) a new medicament or therapeutic target for different pathological conditions such as transplantation or any other pathologies in which immunity is involved, particularly an inappropriate immune response or a chronic immune response is involved, such as autoimmune diseases, infections, cancers, etc.

### Use of New T Cells as a Biomarker

The CD3+CD4+CD57+ILT2+ T cells are particularly useful as a biomarker, especially in a method of prognosis or diagnosis or monitoring of a subject.

In this regard, in a particular embodiment, the invention relates to a method of prognosing or diagnosing or monitoring a subject, comprising determining (in vitro) the presence or level of CD3+CD4+CD57+ILT2+ T cells in a sample from the subject. In a particular embodiment, the method comprises determining (in vitro) the presence or level of said cells in a sample from the subject at two time intervals, or more.

CD3+CD4+CD57+ILT2+ T cells are particularly useful as a biomarker for prognosis, diagnosis or monitoring of pathological conditions associated with inappropriate immune response or in a subject suspected of having a pathological condition associated with inappropriate immune response.

### Pathologies

Pathologies that may be prognosed, diagnosed or monitored by using the CD3+CD4+CD57+ILT2+ T cell population as a biomarker are thus any pathological conditions associated with inappropriate immune response such as graft rejections, immune-related disorders, autoimmune diseases, infectious diseases or cancers.

In a preferred embodiment, the invention is directed to a method of prognosing or diagnosing or monitoring a subject who is transplanted or is a candidate for transplantation, said method comprising determining the presence or level or amount of CD3+CD4+CD57+ILT2+ T cells in a sample from the subject.

The invention also provides a method for evaluating the risk of rejection (in particular, chronic rejection) of a transplant in a transplant subject, comprising determining, in a biological sample of the transplant subject, the presence or level or amount of CD3+CD4+CD57+ILT2+ T cells, said presence or level or amount being indicative of the risk of transplant rejection.

Preferably, the level or amount of CD3+CD4+CD57+ILT2+ T cells is measured in a sample from the transplant subject at two time intervals after transplantation, wherein an increase in the level of CD3+CD4+CD57+ILT2+ T cells is indicative of a risk of transplant rejection.

In this regard, in a particular embodiment, the method for evaluating the risk of rejection of a transplant in a transplant subject, according to the invention, comprises the following steps:
(i) determining the level or amount of CD3+CD4+CD57+ILT2+ T cells in a biological sample of the transplant subject, taken between 2 and 6 weeks post-transplant, preferably between 3 and 5 weeks post-transplant; and
(ii) determining the level or amount of CD3+CD4+CD57+ILT2+ T cells in a biological sample of the transplant subject, taken between 11 and 13 months post-transplant, an increase between the two levels of (i) and (ii) being indicative of a risk of transplant rejection.

Preferably, in step (i) the level of CD3+CD4+CD57+ILT2+ T cells is determined in a biological sample of the transplant subject taken approximately 1 month post-transplant; and in step (ii) the level of CD3+CD4+CD57+ILT2+ T cells is determined in a biological sample of the transplant subject taken approximately 12 months post-transplant.

The present invention may thus be used to predict chronic rejection in transplant patients, thus allowing to identify early at-risk patients, and adapt the immunosuppressive treatments to the risk of rejection of a transplant.

The invention thus also provides a method for evaluating the risk of rejection of a transplant in a transplant subject, that allows to further adapt the subject's post-transplant treatment according to the risk of rejection of a transplant, comprising measuring the presence, amount or absence of CD3+CD4+CD57+ILT2+ T cells in a sample from the subject.

In a particular embodiment, the invention thus relates to a method, wherein the subject's post-transplant treatment is adapted to the risk of rejection of a transplant.

In a particular embodiment, the invention is directed to lung transplantation. In this regard, the invention provides a method for evaluating the risk of rejection of a lung transplant in a lung transplant subject or a candidate, comprising determining, in a biological sample of the lung transplant subject, the presence or level or amount of CD3+CD4+CD57+ILT2+ T cells, said presence or level or amount being indicative of the risk of lung transplant rejection.

In a specific embodiment, the method according to the invention uses CD3+CD4+CD57+ILT2+ T cells as a biomarker in a field of pulmonary transplantation (Tx) in humans, in order to predict Chronic Lung Allograft Dysfunction (CLAD) in a lung transplant subject. Preferably, CD3+CD4+CD57+ILT2+ T cells are used as a noninvasive blood biomarker predictive test for CLAD.

In this regard, the inventors have surprisingly found that the CD3+CD4+CD57+ILT2+ T cell population increases especially during the first year post-lung transplantation and is significantly associated with a risk of lung transplant rejection and the subsequent occurrence of CLAD. In particular, the inventors have interestingly demonstrated the association of an increase of CD3+CD4⁺CD57⁺ILT2⁺ between the measurements at 1 month post-transplantation and those at 12 months in patients who developed a CLAD between 1 and 3 years post-transplantation (ratio 2.6+/−3.4), compared to no increase in the stable group (STA) (ratio 0.8+/−1.3, p=0.01) (as shown in Table 2 of the experimental data below).

The inventors have also demonstrated that the identification of an increase of the CD3+CD4+CD57+ILT2+ T cell population in a blood sample from transplant subjects, between two time intervals after transplantation (i.e., between 1 month post-transplant and 12 months posttransplant), allows to adapt the treatment of the transplant subjects identified as being at risk of lung transplant rejection, and, in consequence, to anticipate the occurrence of chronic lung graft rejection such as CLAD.

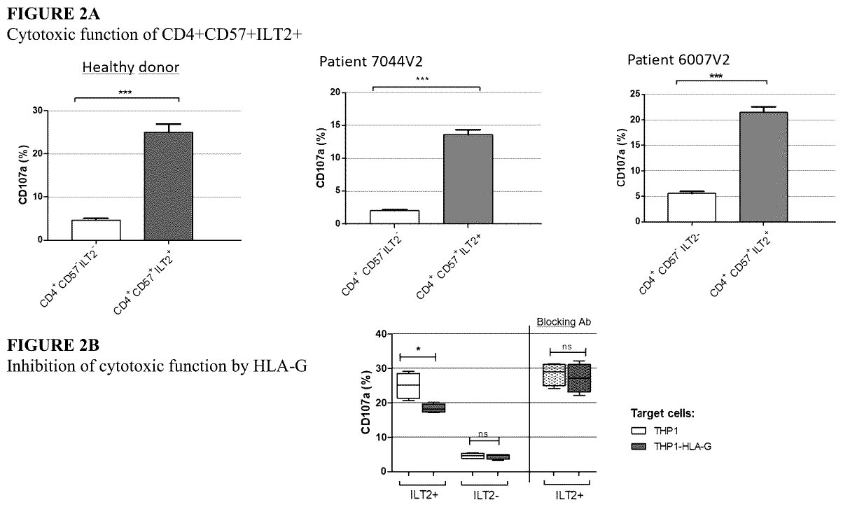

Interestingly, the inventors have also demonstrated that CD3+CD4⁺CD57⁺ILT2⁺ T cells have highly cytotoxic properties (FIG. 2). Such cytotoxic properties could be directed towards the graft, which would explain why an increase of the CD3+CD4⁺CD57⁺ILT2⁺ T cells is associated with graft rejection such as CLAD. The inventors have also shown that the cytotoxic functions of CD3+CD4⁺CD57⁺ILT2⁺ T cells can be inhibited by the expression of HLA-G by their cellular targets (ILT2 being an inhibitory receptor for the HLA-G molecule).

In other particular embodiments, the method according to the invention is used for prognosing or diagnosing or monitoring a subject who has another pathological condition associated with inappropriate immune response, such as, e.g., an autoimmune disease, infectious disease or cancer, and wherein determining the presence or level or amount of CD3+CD4+CD57+ILT2+ T cells in a sample from the subject is indicative of the presence or aggravation of said disease.

In this regard, the invention provides a method for prognosing or diagnosing or monitoring a subject who has a cancer, said method comprising determining the presence or level or amount of CD3+CD4+CD57+ILT2+ T cells in a sample from the subject.

In another embodiment, the invention relates to a method for prognosing or diagnosing or monitoring a subject who has an autoimmune disease, said method comprising determining the presence or level or amount of CD3+CD4+CD57+ILT2+ T cells in a sample from the subject.

In another embodiment, the invention relates to a method for prognosing or diagnosing or monitoring a subject who has an infectious disease, said method comprising determining the presence or level or amount of CD3+CD4+CD57+ILT2+ T cells in a sample from the subject.

In a particular embodiment, the level or amount is determined once. The mere presence (or absence) of said cells, or their level, may indeed be sufficient in itself to determine the patient status.

In another embodiment, the level or amount is determined at least twice at time intervals. Such embodiment allows determination of a variation in the level over time, wherein an increase is indicative of disease progression. In this respect an increase designates any increase of e.g., 10% between the two determinations, preferably at least 15%, or more.

### Samples

The sample according to the invention may be any sample of biological fluid or tissue from a subject, preferably a sample of blood, urine, or saliva. Before subjecting the sample to the test according to the invention, the sample may be pretreated in accordance with standard processing techniques known in the art for each type of the collected sample (such as blood, urine or saliva). The sample may also be aliquoted and stored in a glass or plastic container, e.g., a tube or syringe, etc., without or with appropriate preservative. The sample may be stored in a 4° C. refrigerator for short-term storage. The sample may also be frozen in a −80° C. freezer for long-term storage, as long as necessary for carrying out the method according to the invention.

### Detection Techniques

The presence or level or amount of CD3+CD4+CD57+ILT2+ T cells in a sample may be determined by using standard techniques known per se in the art, such as flow cytometry or various immunodiagnostic assays such as immunoassay (e.g., enzyme immunoassay ELISA, a sandwich immunoassay, a ligand binding assay), radioassay, radioimmunoassay (RIA), enzymatic assay, immunoelectrophoresis or immunoprecipitation, etc.

The detection methods generally include revealing labels such as fluorescent, chemiluminescent, radioactive, enzymatic labels or dye molecules, or other reactions for detecting the formation of a complex between the antigen and the antibody or antibodies reacted therewith.

In a preferred embodiment, determination of the amount of CD3+CD4+CD57+ILT2+ T cells is carried out by flow cytometry. The basic principle of flow cytometry is the passage of cells in single file in front of a laser so they can be detected, counted and sorted. Cell components are fluorescently labelled and then excited by the laser to emit light at varying wavelengths. The fluorescence can then be measured to determine the amount and type of specific T cells present in a sample.

For example, determination of the amount of CD3+CD4+CD57+ILT2+ T cells may be carried out by flow cytometry using any monoclonal antibody anti-CD3, anti-CD4, anti-CD57 and anti-ILT2 known in the art. In a particular embodiment, the monoclonal antibodies according to the invention are antibodies listed in Table 1 below.

TABLE 1
Monoclonal antibodies that may be
used for flow cytometry experiments

| Marker | Supplier | Clone |
|---|---|---|
| CD3 | Miltenyi | REA613 |
| CD4 | Miltenyi | REA623 |
| CD57 | Miltenyi | REA769 |
| ILT2 | eBioscience | HP-F1 |

Other monoclonal antibodies against the specific markers are available in the art and may be used in the invention as well.

For use in the invention, the monoclonal antibodies may be conjugated to fluorochromes such as e.g., PerCP, Vio700, VioBright, FITC, APC, or Vio770, or combinations thereof.

In a particular embodiment, the anti-CD3 monoclonal antibody is conjugated to PerCP-Vio700 fluorochromes.

In a particular embodiment, the anti-CD4 monoclonal antibody is conjugated to VioBright-FITC fluorochromes.

In a particular embodiment, the anti-CD57 monoclonal antibody is conjugated to APC-Vio770 fluorochromes.

In a particular embodiment, the anti-ILT2 monoclonal antibody is conjugated to APC fluorochrome.

### Detection Kits

CD3+CD4+CD57+ILT2+ T cells may be detected with detections kits. In this respect, the invention also relates to kits suitable for detecting said cells. Preferably, the kits of the invention comprise at least an ILT2 binding reagent and a CD57 binding reagent, preferably packaged in different containers. Preferably, the binding reagents are antibodies, such as monoclonal antibodies. Specific examples of such antibodies are listed in Table 1 above.

Other antibodies may be found in the art, such as ChAglyCD3, OKT3, SP-34, UCHT1, RPA-T4, GK1.5, zanolimumab, TB01, NK-1, HNK-1, VMP55, HP-F1, etc.

The detection kit may comprise a combination of antibodies specific for surface markers CD3, CD4, CD57 and ILT2. The kit may further comprise reagents for an immunological detection test and, possibly, an instruction manual.

### Use of New T Cells as a Medicament

The CD3+CD4+CD57+ILT2+ T cells may also be used as a medicament, particularly in a method of treatment of a subject having a pathological condition associated with inappropriate immune response. Such a pathological condition may be any condition, wherein immunity, and in particular, a chronic immune response is involved. The pathological immune disorder may be selected, for example, from autoimmune diseases, infectious diseases and cancers. Indeed, the inventors have demonstrated that CD3+CD4⁺CD57⁺ILT2⁺ T cells are cytotoxic. Such cytotoxic functions of the CD3+CD4⁺CD57⁺ILT2⁺ T cells population may be very useful:
in autoimmune diseases therapy to target self-antigens,
in infectious diseases therapy to target various pathogens (e.g., viruses, bacteria, fungi and parasites), and
in cancer therapy to target tumor cells in order to enhance tumor rejection and inhibit tumor recurrences.

In this regard, the invention provides a method of treatment of an immune disorder selected from an autoimmune disease, an infectious disease or a cancer, comprising administering to a subject in need thereof, a therapeutically effective amount of CD3+CD4+CD57+ILT2+ T cells.

The invention also relates to CD3+CD4+CD57+ILT2+ T cells for use for treating a disorder selected from an autoimmune disease, an infectious disease or a cancer.

The invention also provides a composition comprising CD3+CD4⁺CD57⁺ILT2⁺ T cells, and a pharmaceutically acceptable excipient or carrier, which maybe any conventional pharmaceutically acceptable excipient or carrier known in the art. These pharmaceutically acceptable excipients or carriers may be, for example, inert diluents or fillers (e.g., sucrose, microcrystalline cellulose, starches including potato starch, calcium carbonate, sodium chloride, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., acacia, alginic acid, sodium alginate, gelatin, starch, pregelatinized starch, microcrystalline cellulose, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., stearic acid, silicas, or talc). Other pharmaceutically acceptable excipients can be colorants, flavoring agents, plasticizers, humectants, buffering agents, and the like. Formulation as an aqueous suspension may provide the active ingredient in a mixture with a dispersing or wetting agent, suspending agent, and one or more preservatives. Suitable suspending agents are, for example, sodium carboxymethylcellulose, methylcellulose, sodium alginate, and the like.

The pharmaceutical composition comprising CD3+CD4⁺CD57⁺ILT2⁺ T cells may also comprise any medium or any buffer solution known in the art, which is appropriate for T cells.

Such a composition may also comprise additional active ingredients.

Pharmaceutical compositions comprising CD3+CD4+CD57+ILT2+ T cells may be prepared according to any protocol known in the art for the preparation of cells for therapeutic use, and especially any method of preparation and activation of T cells for an immunotherapeutic treatment. For example, the CD3+CD4⁺CD57⁺ILT2⁺ T cells preparation can be an autologous cell preparation or an allogenic cell preparation or a genetically engineered cell preparation (e.g., using chimeric antigen receptor CAR T-cells). The CD3+CD4+CD57+ILT2+ T cells according to the invention can be isolated or enriched, and thus enriched preparation can be used for the treatment method according to the invention. Preferably, CD3+CD4⁺CD57⁺ILT2⁺ T cells are purified from a blood by Ficoll.

The medicament or the composition comprising CD3+CD4⁺CD57⁺ILT2⁺ T cells according to the invention, may be administered to the subject in need thereof by any route of administration possible for T cells, preferably intravenously or intratumorally.

### Use of New T Cells to Develop New Medicaments

The inventors also propose to use CD3+CD4+CD57+ILT2+ T cells to develop new medicaments, e.g., suitable for treating various pathological conditions wherein immunity, and in particular, a chronic immune response is involved, such as, for example, graft rejection, autoimmune diseases, infectious diseases or cancers, etc.

In a specific embodiment, the invention relates to a method for producing or selecting modulators of CD3+CD4+CD57+ILT2+ T cells, the method comprising the following steps: (i) exposing CD3+CD4+CD57+ILT2+ T cells (in vitro) to a test molecule or treatment, and (ii) determining whether the test molecule modulates an activity of said cells, preferably a cytotoxic activity of said cells. The modulators of T cells may be inhibitors or activators.

A further object of the invention resides in a modulator of CD3+CD4+CD57+ILT2+ T cells for use as a medicament, particularly for the treatment of an immune disorder or inappropriate immune response.

Such modulators of CD3+CD4+CD57+ILT2+ T cells may in particular be used for the preparation of a medicament to reduce the risk of transplant rejection in a transplant patient, or for the treatment of immune conditions such as autoimmune diseases, infections, cancers.

A further object of the invention resides in a use of an inhibitor or depletion of CD3+CD4+CD57+ILT2+ T cells for the preparation of a medicament to reduce the risk of transplant rejection.

In this regard, the medicament for treating graft rejection and other immune pathological conditions, may be prepared based on depletion of CD3+CD4+CD57+ILT2+ T cells. The T cells may be depleted from serum by various techniques known per se in the art, such as immunological, chromatographic, and/or heating/precipitating techniques.

Further aspects and advantages of the invention shall be disclosed in the following experimental section, which illustrates the claimed invention.

### EXAMPLES

### Example I: Association of an Increase in CD3+CD4+CD57+ILT2+ T Cells in the 1st Year Post-Transplantation with an Increased Risk of CLAD 3 Years Post Lung Transplantation

### Patients and Study Design

Blood samples were selected from LTx patients included in the multicentric longitudinal cohort COLT (Cohort in Lung Transplantation, NCT00980967). The cohort COLT included 11 French lung transplantation centers (Programme transplantation 2008, PRTP-13, ClinicalTrials.gov Identifier: NCT00980967).

Pulmonary function tests, clinical data and blood samples were collected at 1-month post-transplantation and every six months post-transplantation during three years post-LTx. Clinical data collection allowed for diagnosis of chronic allograft dysfunction (CLAD) during follow-up at 3 years post-LTx, according to International Society for Heart and Lung Transplantation (Verleden et al., 2019). CLAD diagnosis was made in case of bronchiolitis obliterans syndrome (BOS) or restrictive allograft syndrome (RAS) onset. BOS was defined by a decrease of at least 20% of the maximum FEV1 value recorded after transplantation, according to ISHLT criteria (Verleden et al., 2019). RAS (Restrictive allograft syndrome) or mixed pattern was defined according to recent definition of ISHLT (Glanville et al., 2019), or by previous proposed criteria (Verleden et al., 2011).

Stable graft function (STA), BOS, and RAS status at 3 years-post-LTx were confirmed by repeated sessions of an ad-hoc adjudication multidisciplinary committee using pulmonary function tests, computerized tomography, and after elimination of confounding factors according to the ISHLT practice guidelines (Verleden et al., 2019). All included patients were followed-up at least 3 years after inclusion in COLT study, and classified according to their 3-years post-Tx functional status as follows: stable patients (STA group) and patients with CLAD (CLAD group).

Inclusion criteria of patients in this study were as follows: (i) blood sample(s) at 1 and/or 6, and/or 12 months post-Tx, and (ii) date of CLAD diagnosis after 12 months post-Tx in patients who developed CLAD within the 3 years post-Tx, so that all the patients in the following analysis display normal graft function at all time-points of cells analysis, prior to CLAD onset in the CLAD group. One hundred fifty patients from the COLT cohort were enrolled. These patients of the COLT study were selected according to the availability of blood samples.

### Results

In this example, we studied the blood lymphocytes of lung-transplanted patients included in a multicenter cohort (Cohort for Lung Transplantation, COLT). We had access to at least two blood samples from each patient, obtained at two different clinical checkups during the 1st year (at 1, 6, or 12 months post-transplantation).

All patients studied were stable at 1 year post-transplantation. They were subsequently classified according to their status at 3 years post-transplant into a «stable at 3 years» group (n=31), and a «CLAD» group (n=28, i.e. patients who developed a CLAD between 1 and 3 years post-transplantation). For each time point, we measured the proportion of CD3+CD4+ T cells that expressed both CD57 and ILT2 (CD3+CD4+CD57+ILT2+ T cells).

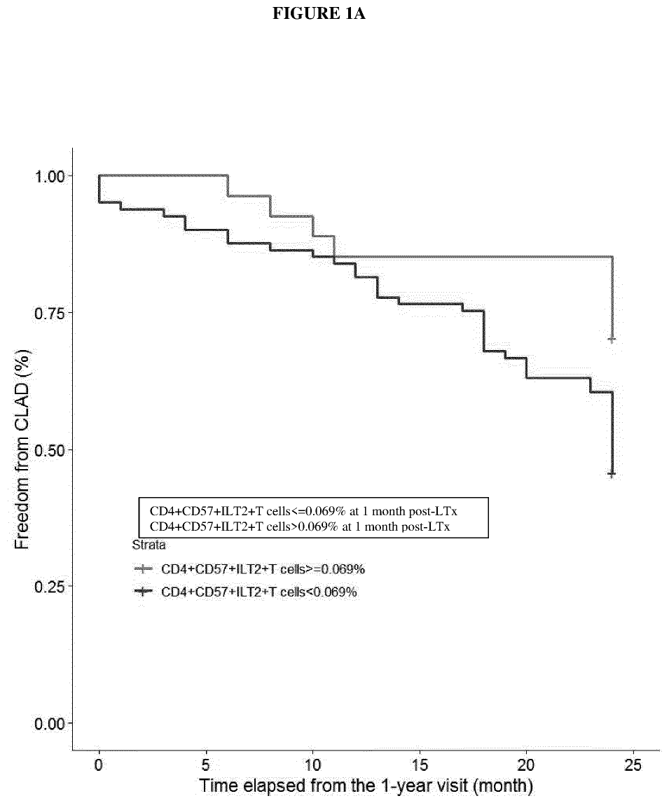

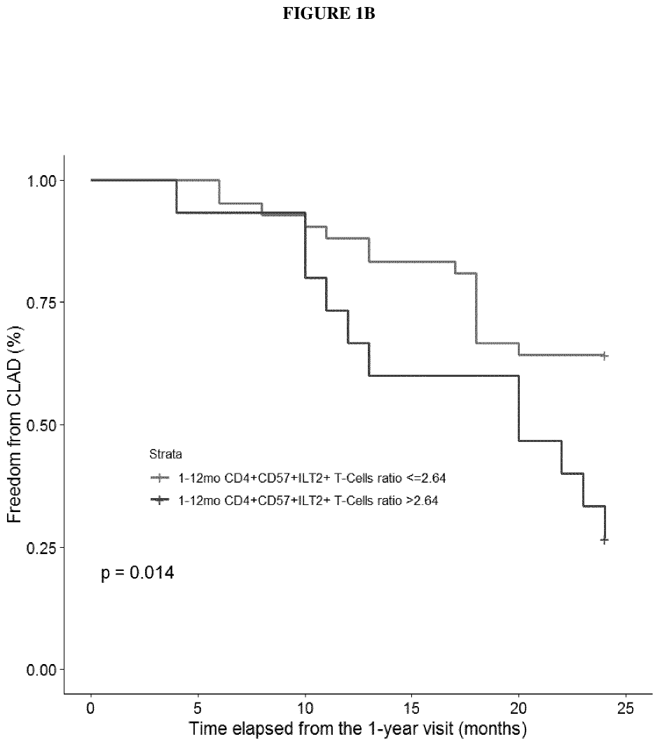

We first looked at the incidence of CLAD according to a cut-off of mean % of CD3+CD4+ILT2+CD57+ T cells at 1-month post-LTx (< or ≥ first IQR (25%), among the 107 patients with available 1-mo Time-point. At 1-mo post-LTx, patients with a mean % of CD3+CD4+CD57+ILT2+ T cells (% of CD4 T cells)≤0.069% had a lower incidence of CLAD at 3 years post-Tx, versus those >0.069% (FIG. 1A, Log-rank test, p=0.04).

We also looked at the freedom from CLAD according to increase of 1-12 months CD3+CD4+ILT2+CD57+ T cells RATIO. CLAD developed in 26 patients (46%) during follow-up of patients with available 1-12 months CD3+CD4+ILT2+CD57+ T cells ratio. 3 patients died during follow-up until 3 years post-LTx, but did not develop CLAD before death and were simply censored for assessment of freedom from CLAD. Freedom from CLAD was lower in patients with 1-12 months CD3+CD4+ILT2+CD57+ T cells ratio >2.64 (n=15, 26% of patients) than in patients with ratio ≤2.64 (n=42): 3 year-freedom frequencies were 27% (95% CI: 8%-50%) versus 64% (95% CI: 48%-77%) (log-rank, p=0.014) (FIG. 1B).

49 other cellular populations were also measured, including the CD4+ T cells expressing CD57 with no regard to ILT2 co-expression (CD4+CD57+ T cells), CD4+ T cells expressing CD57 but negative for ILT2 expression (CD4+CD57+ILT2-neg T cells), and the well-described regulatory subset of TREG cells (CD4+CD25+CD127lo T cells).

Among these 49 peripheral cell populations, the most striking result was the association of an increase of CD3+CD4+CD57+ILT2+ between the measurements at 1 month post-transplantation and those at 12 months in patients who developed a CLAD between 1 and 3 years post-transplantation (ratio 2.6+/−3.4), compared to no increase in the stable group (ratio 0.8+/−1.3, p=0.01) (Table 2 below).

This association was observed only for the CD3+CD4+CD57+ILT2+ population, and not for the CD3+CD4+CD57+ population or the CD3+CD4+CD57+ILT2-neg population, or any other population studied.

TABLE 2

| | Stable (STA) | CLAD | |
|---|---|---|---|
| Cell population | (n = 31) | (N = 28) | p |
| Increase ratio for CD3 + | 0.8 (1.3) | 2.6 (3.4) | 0.01 |
| CD4 + CD57 + ILT2 + | | | |
| T cells between 1 and 12 months | | | |
| post transplantation | | | |
| (% at 12 months)/(% at 1 month) | | | |

### Conclusion

Hence, at 12 months, CD3+CD4+ILT2+CD57+ T cells ratio discriminate subsequent status of CLAD and STA patients at 3 years post-Tx. In conclusion, ILT2 expression seems a key feature of differentiated cytotoxic CD3+CD4+CD57+ILT2+ T cells, potentially rendering them susceptible to HLA-G-mediated inhibition. CD3+CD4+CD57+ILT2+ T cells are increased in peripheral blood of LTx-recipients at risk of subsequent CLAD onset, whereas HLA-G is infrequently neo-expressed in the graft of such at-risk patients, which could reflect an ineffective attempt to stop the local process of rejection. Our data show that early increase of peripheral CD3+CD4+CD57+ILT2+ T cells in LTx recipients may be a predictor of subsequent CLAD onset, and could reflect a graft-infiltrating CD3+CD4+CD57+ILT2+ T cells as a differentiated cytotoxic population involved in CLAD development.

### Example II: Univariate and Multivariate Analysis of the Risk of CLAD at 3 Years Post-Transplant (Cox Model)

The association of clinical, functional, histologic, and immunologic factors with CLAD onset and graft loss were assessed in univariate analysis and backward-elimination multivariate Cox regression analyses in n=107 patients with available % of different lymphocyte subpopulations at 1-month post-LTx.

### Statistics

Continuous variables are described with mean (SD), or median (range), and were compared by Student t test or Mann-Whitney U-test. Categorical variables are described number (%) and were compared by chi-square test or Fisher' exact tests.

Time to CLAD onset (freedom from CLAD) and time to death or retransplantation (graft survival) were estimated by the Kaplan-Meier estimator and compared by log-rank test.

Univariate and multivariable Cox models were built to assess the association between 20% of cells subpopulations measured at time-point 1-month post-LTx or 1-12 months T-cells ratio and freedom from CLAD survival and graft survival, with adjustment for potential confounding factors. Factors associated with freedom from CLAD and graft survival by univariate analyses (at a significance of p<0.2) were selected for multivariate analyses. Among cells populations, CD3+CD4+CD25+CD127low, CD8+CD25+, and CD3+CD4+CD57+ILT2+ T cells assessed at Time-point 1-month post-LTx and 1-12 months, CD3+CD4+CD57+ILT2+ cells ratio were included in the Cox model. For univariate and multivariable Cox models built to assess the association between 1-12 months T-cells ratio and freedom from CLAD survival and graft survival, analysis was reduced to n=57 patients with at least available different 2 time-point sampling during follow-up. Graft survival, freedom from CLAD and follow-up were computed from the time of CD3+CD4+CD57+ILT2+ cells ratio calculation in all patients. The last follow-up date for all survival models was 3 years post-LTx. The proportional hazards assumption was tested using Grambsch and T. M. Therneau tests based on weighted residuals (cox.zph function in R) (Grambsch, 1994). Quantification of follow-up was done using the Kaplan-Meier estimate of potential follow-up (Schemper, 1996). For all analyses, p<0.05 was considered statistically significant. Analyses involved the XXXX of Stata v12 for Macintosh (StataCorp LP, College Station, TX).

### Results

The “CLAD-freed survival” is defined by the time interval between month 12 post transplantation (date of the measurement of the month-12/month-1 ratio for the CD3+CD4+CD57+ILT2+ population, and the occurrence of CLAD. Survival without CLAD was analyzed by the Kaplan-Meier method and compared by log-rank test in pulmonary transplant patients with multiple samples available in the first year post-transplant (n=59). Uni- and multivariate Cox models were used to evaluate the association between the ratio of increase of the CD3+CD4+CD57+ILT2+ T lymphocyte population during the first year post-transplantation and CLAD-free survival at 3 years. Factors associated with the occurrence of a CLAD identified in univariate analysis with a significance of p<0.2 were selected for multivariate analysis, including graft ischemia duration, CMV mismatch, induction therapy post-Tx. Multivariate analysis showed that only the ratio of increase of CD3+CD4+CD57+ILT2+ T lymphocytes at 1 year post-transplant was significantly associated with the occurrence of CLAD at 3 years (Log-rank test, p=0.002), with a hazard ratio (HR) of 1.24 (95% confidence interval (CI): [1.08-1.42]) for each 0.1-fold increase in the 1-year post-transplant increase ratio. It is important to note that no significant association was found when the CD3+CD4+CD57+ T cell population as a whole. Only when ILT2 was added as an additional discriminative parameter to this population, did we observe a technical advantage in predicting CLAD.

### Example III: Cytotoxicity Assays

We studied the functions of such CD3+CD4+CD57+ILT2+ T cells from the same COLT cohort patients (as described in Example I above) using cytotoxicity assays which were CD107a degranulation assays.

CD107a is a lysosome-associated membrane glycoprotein (LAMPs) that is expressed at the surface of immune cells upon cytolytic degranulation. The percentage of CD107a-expressing-cells at the end of the assay was used as a marker of cytolytic-degranulation, and thus of cytotoxic-killing of the target cells. The Effector:Target ratio used here was PBMC:Target of 2:1 in each well.

The monocytic cell line THP1 (ATCC) was used as target cells facing either CD3+CD4+CD57−ILT2− or CD3+CD4+CD57+ILT2+ effector cells from healthy donor or from two patients LTx 7044V2 and LTx 6007V2 (FIG. 2A).

THP1 Target cells were transduced, or not, to express membrane-bound HLA-G1 (THP1 and THP1-HLA-G1 cells), as previously described (Dumont et al. Cancer Immunology Research, 2019).

THP1 and THP1-HLA-G1 cells were used as target cells facing either CD3+CD4+CD57-ILT2− or CD3+CD4+CD57+ILT2+ effector cells (FIG. 2B).

Then, target cells were coated for 15 minutes with anti-CD3 mAb (50 ng/ml; clone OKT3, Orthoclone) on ice. PBMCs were incubated at 106/mL with polyclonal immunoglobulin and then for 20 minutes at 37° C. with 20 μg/mL of a blocking anti-ILT2 antibody (clone GHI/75, BioLegend) (FIGS. 2A and 2B), or a control antibody in inhibition experiments (FIG. 2B).

100 μL of PBMCs were, then directly added to the OKT3-coated THP1/THP1-HLA-G1 target cells in culture medium supplemented with monensin and brefeldin A (Protein Transport Inhibitor Cocktail, eBioscience) or Cell Stimulation Cocktail (eBioscience), in the presence of PE-conjugated anti-CD107a antibody (clone H4A3, BioLegend) or isotype control. After 4 h of co-incubation at 37° C., cells were washed and stained for flow cytometry analysis. Acquisition was performed on a MACS Quant10 flow cytometer (Miltenyi Biotec) and data analysis on FlowJo10 Software. The cytolytic degranulation by CD3+CD4+CD57+ILT2+ or CD3+CD4+CD57−ILT2− T cell subsets were evaluated using the percentage of CD107a-positive cells, respectively (FIGS. 2A and 2B).

We demonstrated in vitro that CD3+CD4+CD57+ILT2+ T cells are cytotoxic, whereas their ILT2-negative counterparts (CD3+CD4+CD57+ILT2-neg T cells) were not (FIG. 2A). Such cytotoxic functions could be directed towards the graft, which would explain why an increase of the CD3+CD4+CD57+ILT2+ T cells is associated with CLAD.

Finally, because ILT2 is an inhibitory receptor for the HLA-G molecule, we have demonstrated that the cytotoxic functions of CD3+CD4+CD57+ILT2+ T cells can be inhibited by the expression of HLA-G by their cellular targets (FIG. 2B).

### Example IV: Quantifying CD3+CD4+CD57+ILT2+ T Cell Population by Flow Cytometry

The implementation of the invention consists in quantifying by flow cytometry, in the peripheral blood of the patient, the proportion of CD3+CD4+CD57+ILT2+ cells among the CD3+CD4+ subset T lymphocytes from sequential blood samples at 1 month and 12 months post-transplant.

Peripheral blood Mononuclear Cells (PBMCs) obtained from COLT patients (as described in Example I above) at each visit were here analyzed. PBMCs were isolated by Ficoll gradient centrifugation (Ficoll-Paque, LifeSciences), frozen at −80° C. and stored at the Centre de Resources Biologiques (CRB) of Nantes. After thawing, cells were washed and incubated for 1 h at 37° C. In order to avoid any non-specific binding of the labeling antibodies, Fc receptors were blocked with polyclonal human Ig. A FcR blocking step is performed for 5 min (polyclonal human Ig FcR blocking reagent, Miltenyi Biotec). Cells were then labeled with antibodies targeting cell-surface markers from B cells, CD4+ and CD8+ T cells, and antigen presenting cells, which allow us to characterize a total of 49 cell populations. In parallel, PBMC from healthy individuals (EFS Saint-Louis hospital, Paris, France) were analyzed as controls.

The following monoclonal antibodies (mAb) or combinations thereof (as described in Table 3 below) were used for cell-surface staining of PBMC (15 min) and analysis: CD3 PerCP-Vio700 (Miltenyi Mix), CD4 Vio Bright FITC (Miltenyi Mix), CD8 PE-Vio (Miltenyi Mix), CD19 VG (Miltenyi Mix), ILT2 APC (eBioscience), CD57 APC-Vio770 (Miltenyi), PD1 BV421 (Biolegend) to target T and B lymphocyte populations; Miltenyi mix, ILT2 APC (eBioscience), CD25 eFluor450 (eBioscience), CD127 PE-Vio770 (Miltenyi) to identify regulatory peripheral cells; and CD14 VioBlue (Miltenyi), CD3 PerCP-Vio 700 (Miltenyi), HLA-G FITC (Exbio), HLA-DR PE-Vio770 (Miltenyi), ILT4 APC (Miltenyi), CD83 APC-Vio770 (Miltenyi) to characterize antigen-presenting cells.

TABLE 3
antibodies for cytometry analysis

| | Panel 1 | Panel 2 | Panel 3 |
|---|---|---|---|
| antibodies | CD3 PerCP-Vio700 | CD3 PerCP-Vio700 | CD14 VioBlue |
| | CD4 Vio Bright FITC | CD4 Vio Bright FITC | CD3 PerCP-Vio 700 |
| | CD8 PE-Vio | CD8 PE-Vio | HLA-G FITC |
| | CD19 VG | CD19 VG | HLA-DR PE-Vio770 |
| | ILT2 APC | ILT2 APC | ILT4 APC |
| | CD57 APC-Vio770 | CD25 eFluor450 | CD83 APC-Vio770 |
| | PD1 BV421 | CD127 PE-Vio770 | |

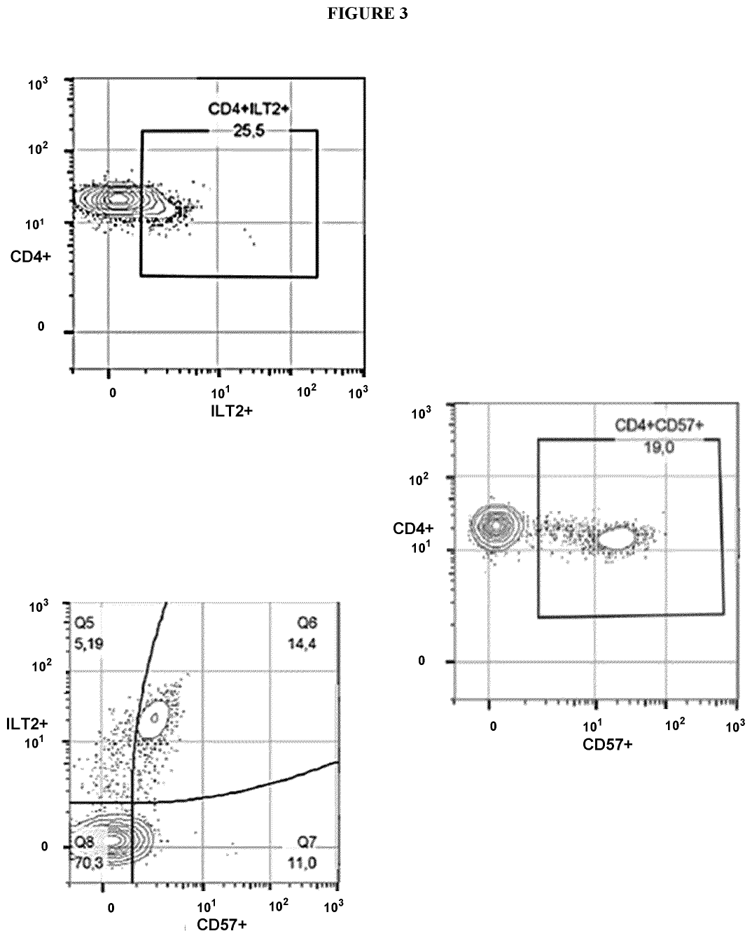

After washing the cells with PBS and centrifugation, the samples were analyzed by flow cytometer (for example, a MACSQuant 10 flow cytometer (Miltenyi Biotech), analyzed using the MACSQuantify software (Miltenyi Biotech) and FlowJo software, using appropriate isotypic controls to account for nonspecific background staining of the cell subsets of interest. A representative example is shown in FIG. 3. These same membrane labelling described in FIG. 3 can be carried out on whole blood directly without a step of isolating the blood cells on Ficoll.

### Conclusion

The increased ratio of CD3+CD4+CD57+ILT2+ T cells 1 year post-transplant, as measured e.g., by flow cytometry, is a non-invasive predictive biomarker for lung transplant rejection, at a time when this rejection process is subclinical and potentially reversible by modification of immunosuppressive treatments.

### REFERENCES

Verleden G M, Glanville A R, Lease E D, Fisher A J, Calabrese F, Corris P A, Ensor C R, Gottlieb J, Hachem R R, Lama V, Martinu T, Neil D A H, Singer L G, Snell G, Vos R. Chronic lung allograft dysfunction: Definition, diagnostic criteria, and approaches to treatment—a consensus report from the pulmonary council of the ISHLT. J Heart Lung Transplant 2019; 38:493-503.
Verleden G M, Vos R, Verleden S E, De Wever W, De Vleeschauwer S I, Willems-Widyastuti A, Scheers H, Dupont L I, Van Raemdonck D E, Vanaudenaerde B M. Survival determinants in lung transplant patients with chronic allograft dysfunction. Transplantation 2011; 92: 703-708.
Weill D, Benden C, Corris P A, Dark J H, Davis R D, Keshavjee S, Lederer D J, Mulligan M J, Patterson G A, Singer L G, Snell G I, Verleden G M, Zamora M R, Glanville A R. A consensus document for the selection of lung transplant candidates: 2014—an update from the pulmonary transplantation council of the international society for heart and lung transplantation. J Heart Lung Transplant 2015; 34:1-15.
Meyer K C, Raghu G, Verleden G M, Corris P A, Aurora P, Wilson K C, Brozek J, Glanville A R, Committee IAEBTF, Committee IAEBTF. An international ishlt/ats/ers clinical practice guideline: Diagnosis and management of bronchiolitis obliterans syndrome. Eur Respir J 2014; 44:1479-1503.
Glanville A R, Verleden G M, Todd J L, Benden C, Calabrese F, Gottlieb J, Hachem R R, Levine D, Meloni F, Palmer S M, Roman A, Sato M, Singer L G, Tokman S, Verleden S E, von der Thusen J, Vos R, Snell G. Chronic lung allograft dysfunction: Definition and update of restrictive allograft syndrome—a consensus report from the pulmonary council of the ishlt. J Heart Lung Transplant 2019; 38:483-492.
Neuringer I P, Chalermskulrat W, Aris R. Obliterative bronchiolitis or chronic lung allograft rejection: A basic science review. J Heart Lung Transplant 2005; 24:3-19.
Salman J, Ius F, Knoefel A K, Sommer W, Siemeni T, Kuehn C, Tudorache I, Avsar M, Nakagiri T, Preissler G, Hatz R, Greer M, Welte T, Haverich A, Warnecke G. Association of higher cd4(+) cd25(high) cd127(low), foxp3(+), and il-2(+) t cell frequencies early after lung transplantation with less chronic lung allograft dysfunction at two years. Am J Transplant 2017; 17:1637-1648.
Meloni F, Morosini M, Solari N, Bini F, Vitulo P, Arbustini E, Pellegrini C, Fietta A M. Peripheral cd4+cd25+ treg cell expansion in lung transplant recipients is not affected by calcineurin inhibitors. Int Immunopharmacol 2006; 6:2002-2010.
Durand M, Lacoste P, Danger R, Jacquemont L, Brosseau C, Durand E, Tilly G, Loy J, Foureau A, Royer P J, Tissot A, Roux A, Reynaud-Gaubert M, Kessler R, Mussot S, Dromer C, Brugiere O, Mornex J F, Guillemain R, Claustre J, Degauque N, Magnan A, Brouard S, Colt, Sys C C. High circulating cd4(+)cd25(hi)foxp3(+) t-cell sub-population early after lung transplantation is associated with development of bronchiolitis obliterans syndrome. J Heart Lung Transplant 2018; 37:770-781.
Espinosa J, Herr F, Tharp G, Bosinger S, Song M, Farris A B, 3rd, George R, Cheeseman J, Stempora L, Townsend R, Durrbach A, Kirk A D. Cd57(+) cd4 t cells underlie belatacept-resistant allograft rejection. Am J Transplant 2016; 16:1102-1112.
Brugière O, Thabut G, Pretolani M, Krawice-Radanne I, Dill C, Herbreteau A, et al. Immunohistochemical Study of HLA-G Expression in Lung Transplant Recipients. Am J Transplant. juin 2009; 9(6):1427-38.

Brugière O, Thabut G, Krawice-Radanne I, Rizzo R, Dauriat G, Danel C, et al. Role of HLA-G as a Predictive Marker of Low Risk of Chronic Rejection in Lung Transplant Recipients: A Clinical Prospective Study: HLA-G Is Associated With Lung Graft Acceptance. Am J Transplant. févr 2015; 15(2):461-71.
Cosman D, Fanger N, Borges L, Kubin M, Chin W, Peterson L, Hsu M L. A novel immunoglobulin superfamily receptor for cellular and viral MHC class I molecules. Immunity 1997; 7:273-82.
Liang S, Zhang W, Horuzsko A. Human ILT2 receptor associates with murine MHC class I molecules in vivo and impairs T cell function. Eur J Immunol 2006; 36:2457-71.
Navarro F, Llano M, Bellon T, Colonna M, Geraghty D E, Lopez-Botet M. The ILT2(LIR1) and CD94/NKG2A NK cell receptors respectively recognize HLA-G1 and HLA-E molecules co-expressed on target cells. Eur J Immunol 1999; 29:277-83.
Shiroishi M, Tsumoto K, Amano K, Shirakihara Y, Colonna M, Braud V M, Allan D S, Makadzange A, Rowland-Jones S, Willcox B, et al. Human inhibitory receptors Ig-like transcript 2 (ILT2) and ILT4 compete with CD8 for MHC class I binding and bind preferentially to HLA-G. Proc Natl Acad Sci USA 2003; 100:8856-61
Bellon T, Kitzig F, Sayos J, Lopez-Botet M. Mutational analysis of immunoreceptor tyrosine-based inhibition motifs of the Ig-like transcript 2 (CD85j) leukocyte receptor. J Immunol 2002; 168:3351-9.
Naeim Faramarz, Atlas of hematopathology, Chapter T-cell associated CD molecules; ed. Elsevier 2013.
Saverino D, Fabbi M, Ghiotto F, Merlo A, Bruno S, Zarcone D, Tenca C, Tiso M, Santoro G, Anastasi G, et al. The CD85/LIR-1/ILT2 inhibitory receptor is expressed by all human T lymphocytes and down-regulates their functions. J Immunol 2000; 165:3742-55.
Naji A, Menier C, Morandi F, Agaugué S, Maki G, Ferretti E, Bruel S, Pistoia V, Carosella E D, Rouas-Freiss N. Binding of HLA-G to ITIM-bearing Ig-like transcript 2 receptor suppresses B cell responses. J Immunol 2014.
Dietrich J, Cella M, Colonna M. Ig-like transcript 2 (ILT2)/leukocyte Ig-like receptor 1 (LIR1) inhibits TCR signaling and actin cytoskeleton reorganization. J Immunol 2001; 166:2514-21.
Grambsch P M T T. Proportional hazards tests and diagnostics based on weighted residuals. Biometrika 1994; 81: 551-526.
Schemper M, Smith T L. A note on quantifying follow-up in studies of failure time. Control Clin Trials 1996; 17: 343-346.

1.-19. (canceled)

20. A method of monitoring a subject, comprising determining in vitro the presence or level of CD3+CD4+CD57+ILT2+ T cells in a sample from the subject.

21. The method of claim 20, comprising determining the presence or level of said cells in a sample from the subject at two time intervals, or more.

22. The method of claim 20, wherein the subject is transplanted or is a candidate for transplantation.

23. The method of claim 20, wherein the subject has a cancer.

24. The method of claim 20, wherein the subject has an autoimmune or infectious disease.

25. The method of claim 20, wherein the sample is a sample of biological fluid or tissue, said biological fluid being a sample of blood, plasma, serum, urine, or saliva.

26. The method of claim 20, wherein the presence or level of CD3+CD4+CD57+ILT2+ T cells in a sample is determined by flow cytometry, radioassay, enzymatic assay, immunoassay or radioimmunoassay.

27. A method for evaluating the risk of rejection of a transplant in a transplant subject, comprising determining, in a biological sample of the transplant subject, the level of CD3+CD4+CD57+ILT2+ T cells, said level being indicative of the risk of transplant rejection.

28. The method of claim 27, wherein the transplant is a lung transplant.

29. The method of claim 27, wherein an increase in the level of CD3+CD4+CD57+ILT2+ T cells, in the subject after the transplant, is indicative of a risk of transplant rejection.

30. The method of claim 27, comprising:
(i) determining the level of CD3+CD4+CD57+ILT2+ T cells in a biological sample of the transplant subject, taken between 2 and 6 weeks post-transplant; and
(ii) determining the level of CD3+CD4+CD57+ILT2+ T cells in a biological sample of the transplant subject, taken between 11 and 13 months post-transplant;
an increase between the two levels of (i) and (ii) being indicative of a risk of transplant rejection.

31. The method of claim 30, wherein in step (i) the level of CD3+CD4+CD57+ILT2+ T cells is determined in a biological sample of the transplant subject taken approximately 1 month post-transplant; and in step (ii) the level of CD3+CD4+CD57+ILT2+ T cells is determined in a biological sample of the transplant subject taken approximately 12 months post-transplant.

32. The method of claim 27, wherein the subject's post-transplant treatment is adapted to the risk of rejection of a transplant.

33. A method for producing or selecting modulators of T cells, comprising exposing CD3+CD4+CD57+ILT2+ T cells to a test molecule or treatment, and determining whether the test molecule modulates a cytotoxic activity of said cells.

34. A composition comprising CD3+CD4+CD57+ILT2+ T cells and an excipient or diluent or culture medium.

35. A method of reducing the risk of transplant rejection in a transplant patient comprising administering an inhibitor of CD3+CD4+CD57+ILT2+ T cells to said patient or depleting CD3+CD4+CD57+ILT2+ T cells present in said patient.

36. A kit comprising an ILT2 binding reagent and a CD57 binding reagent, optionally packaged in different containers.

37. The kit of claim 36, wherein the binding reagents are antibodies.

38. The kit of claim 36, further comprising reagents for an immunological detection test and, optionally, an instruction manual.
